(19) United States
(12) Patent Application Publication
(10) Pub. No.: US 2024/0415611 A1
(43) Pub. Date: Dec. 19, 2024

(54) METHOD FOR ADAPTING AN ORTHODONTIC APPARATUS

(71) Applicant: DENTAL MONITORING, PARIS (FR)

(72) Inventors: Thomas PELLISSARD, MAISONS-ALFORT (FR); Philippe SALAH, PARIS (FR)

(21) Appl. No.: 18/564,222

(22) PCT Filed: May 24, 2022
(86) PCT No.: PCT/EP2022/064126

§ 371 Date: Feb. 28, 2024

(30) Foreign Application Priority Data
May 25, 2021 (FR) FR2105394

(51) Int. Cl. A61C 7/00; G06T 7/00; G16H 30/40

(52) U.S. Cl. CPC A61C 7/002; G06T 7/0012; G16H 30/40

(57) ABSTRACT

The invention relates to a system for adapting an orthodontic apparatus intended for a patient, said system comprising the following steps: -a) determining, at a parameterisation time, by a first dental healthcare professional and specifically for said patient, conditions for communicating updated information relating to the configuration of the patient's dentition; -b) acquiring, at an updated time subsequent to the parameterisation time, by means of an acquisition device and by the patient or an acquaintance of the patient, an extraoral depiction of at least one portion of the patient's dental arches, referred to as an “updated depiction”, said updated depiction consisting of a set of images and/or of a three-dimensional digital model; -c) analysing said updated depiction, so as to determine information regarding said dentition configuration at the updated time, or “updated information”, the updated information consisting of a set of information elements, each expressing a status for a relevant treatment aim in light of the updated depiction; -d) communicating the updated information to at least one second dental healthcare professional under the conditions determined in step a); -e) adapting said orthodontic apparatus on the basis of the updated information communicated in step d).

### TECHNICAL FIELD

The present invention relates to a method for adjusting an orthodontic appliance intended for a patient, in particular by virtue of improved monitoring of the patient's dental configuration.

The expression “dental configuration” is understood to mean
the shape of the upper and lower jaws, which particularly depends on the shape and position of the teeth on these jaws, but also on the general shape of these jaws, and
the relative arrangement of the lower jaw with respect to the upper jaw, in particular in the “closed mouth” position.

### PRIOR ART

The applicant has developed methods allowing the position of a patient's teeth before, during or after an orthodontic treatment to be monitoring remotely.

These methods are based on comparison of photos taken by the patient, at an update time, by means of her or his mobile telephone, with views of a three-dimensional digital model of one of her or his dental arches. More precisely, an initial model of the dental arch is generated at an initial time, conventionally with a 3D scanner, then divided into tooth models. After the patient has acquired the photos, the initial model is deformed, by moving the tooth models, to obtain the best match to the photos. The comparison of the initial models and of the deformed model thus obtained then provides information on the movement of the teeth since the initial time. Since the initial model is very exact, the same advantageously goes for the deformed model, and therefore for the information resulting from said comparison. These methods are particularly described in PCT/EP2015/074868 or PCT/EP2015/074859.

The orthodontist monitoring a patient may thus benefit from many data on the position of the patient's teeth, without the patient having to go anywhere. Typically, several hundred data are provided thereto. The orthodontist may, based on these data, assess whether the treatment objectives have been met and, where appropriate, adjust the orthodontic appliance intended for the patient.

There is a constant need to facilitate and make this adjustment of the orthodontic appliance more exact.

One objective of the present invention is to meet this need, at least in part.

### SUMMARY OF THE INVENTION

According to a first main aspect, the invention provides a method for adjusting an orthodontic appliance intended for a patient, said method comprising the following steps:
a) at a parameterization time, parameterization, by a first dental professional and specifically for said patient, of conditions for communication, by computer, of updated information relating to the patient's dental configuration;
b) at an update time subsequent to the parameterization time, acquisition, by means of an acquiring device and by the patient or a friend or relative of the patient, of a representation, preferably an extraoral representation, of at least one portion of the patient's dental arches, which is referred to as the “updated representation”, said updated representation preferably consisting of a set of “updated” images and/or of a three-dimensional digital model, or “updated model”;
c) analysis of said updated representation, by computer, so as to determine information on said dental configuration at the update time, or “updated information”, the updated information preferably consisting of a set of information elements each expressing a status determined, with regard to the updated representation, for a respective treatment objective, a status in particular possibly indicating that the treatment objective is monitored and/or has been reached and/or has not been reached at the current time;
d) communication, preferably on a screen, of the updated information to at least one second dental professional, under the conditions determined in step a),
e) preferably, depending on the updated information communicated in step d), either adjusting said orthodontic appliance or not.

As will be seen in greater detail in the remainder of the description, a method according to the invention makes it possible to filter the updated information that is presented to the second dental professional, depending on conditions that she or he has herself or himself set for communication with the patient. The inventors have discovered that presentation of a large amount of data can prove detrimental, in particular to the analysis carried out by the second dental professional, and therefore to the effectiveness, or even the exactitude, of the adjustment of the orthodontic appliance carried out consequently. The inventors have also observed that dental professionals do not always use the same data and that applying similar filtering to the data for every dental professional could therefore be detrimental. Lastly, the inventors observed that applying identical filtering for all patients of a dental professional is also unsatisfactory, each orthodontic treatment being specific.

According to the invention, at the parameterization time, conventionally at the start of an orthodontic treatment, the first dental professional decides, specifically for the patient in question and depending on her or his own assessment of the patient's dental situation, under which conditions the updated information must be provided. Advantageously, at the update time, the second dental professional, who may in particular be the first dental professional, sees only the updated information that the first dental professional has previously decided to provide thereto. She or he may thus concentrate only on information that she or he deems useful to carrying out the best analysis of the patient's dental situation at the update time. This analysis is advantageously faster and more exact because it is not hindered by information that the first dental professional would not take into consideration anyway.

A method according to the invention may further comprise, in particular, one or more of the following optional features:
preferably, the conditions determined in step a) set, for at least one said treatment objective, preferably for each treatment objective, at least one, or even a single, time range, or “communication range”, exclusively during which the corresponding information element, determined in step c), is communicable in step d);
the conditions determined in step (a) set for at least one said treatment objective,
a minimum or maximum length of time between the update time and at least one reference time, the reference time preferably being a time marking the end of the orthodontic treatment or one portion of the orthodontic treatment or the time of step d) of a prior series of steps b) to e), and/or
a maximum frequency of communication of the information element;
one or more recipients to whom the information element must be communicated;
the conditions determined in step a) set, for said treatment objective, different said communication ranges depending on the status of said treatment objective;
the conditions determined in step a) set, for said treatment objective, a first said communication range for the information element expressing that the treatment objective has been reached, i.e. for the status “objective reached”, and, immediately after said first communication range, a second said communication range for the information element expressing that the treatment objective has not been reached;
the conditions of communication of at least one information element, and preferably of each information element, determined in step a), and in particular the communication ranges, are dependent on the treatment objective in question and/or on the status of the treatment objective expressed by said information element and/or on the recipient of the information element;
the format and/or the content of an information element depends on the recipient of the information element, and for example differ/differs depending on whether this information element is intended for an orthodontist, an orthodontist assistant or the patient;
preferably, the status of a treatment objective, and preferably of any treatment objective, is selected from a group of potential statuses containing less than 10 potential statuses;
preferably, the number of treatment objectives is less than 30;
preferably, for at least one, and preferably for each treatment objective, the updated information contains an evaluation of the time remaining, from the update time, to reach the treatment objective, preferably in compliance with a treatment plan established before the update time, preferably at the parameterization time, and/or contains an indication of the date since which the treatment objective has been monitored;
preferably, at least one of said treatment objectives relates to overbite or overjet;
preferably, the acquiring device is a mobile telephone;
preferably, the updated representation consists of a set of images, referred to as “updated images”;
in step c), the updated representation is submitted to at least one neural network, preferably at least one convolutional neural network;
preferably, at least one second dental professional is different from the first dental professional;
preferably, the parameterization in step a) and the communication in step d) are carried out by means of the same computer interface;
preferably, the interface simultaneously presents on a computer screen:
an identifier of the patient; and
a timeline representing successive times that are individually selectable by an operator, and particularly by the first and second dental professionals; and
for each selectable time selected, information relating to the dental configuration at the selected time, and preferably at least one representation, preferably at least one extraoral representation, of the patient's dental arches at the selected time, or “main representation”, and/or an information element expressing a status, at the selected time, of at least one treatment objective and/or at least one observation relating to the patient's dental configuration at the selected time; and
a treatment-objective-monitoring zone providing information elements expressing statuses of said treatment objectives at the time when the interface is displayed;
in step e), the adjustment of the orthodontic appliance comprises manufacture or modification of an orthodontic appliance intended for the patient;
in step e), prior to the adjustment of the orthodontic appliance, an appointment is made for the patient to see the first and/or the second dental professional, depending on the updated information;
the orthodontic appliance adjusted in step e) is supplied to the patient, and is preferably sent by post, in particular when the orthodontic appliance is an orthodontic aligner;
series of steps b) to e) are repeated at various update times during an orthodontic treatment of a patient, the update times of two successive series preferably being separated by more than one week, more than two weeks and/or less than 2 months, and preferably less than one month.

In step a), the parameterization consists in entering the conditions of communication into a computer. Step c) and step d) are carried out by computer, preferably without human assistance, i.e. automatically. The same preferably goes for the adjustment of the orthodontic appliance in step e).

Steps a), c), and d), and preferably e), are implemented by means of a computer, and preferably carried out by a computer. The computers used for these steps may be identical or different. If they are different, the computers communicate with one another: in particular, the computer parameterized in step a) transfers the conditions of communication to the computer that defines the updated information to be presented in step d) and to the computer that communicates the updated information in step d). The computer that analyzes said updated representation in step c) transmits the updated information to the computer that communicates it in step d). Furthermore, the acquiring device transmits the updated representation to the computer that analyzes it in step c).

The invention thus also relates to:
a computer program comprising program-code instructions for implementing steps a), c), d) and preferably e);
a data medium on which such a program is stored, for example a memory or a CD-ROM,
a computer in which such a program is loaded.

The invention also relates to a system for implementing a method according to the invention, said system comprising:
a first computer, parameterized, in step a), by the first dental professional to define the conditions of communication of the updated information;
an acquiring device, preferably a mobile telephone, configured to acquire said updated representation in step b);
a second computer, identical to or different from the first computer
   able to receive from the acquiring device and to analyze said updated representation to determine the updated information in step c),
   able to receive the conditions of communication from the first computer, and
   able to present the updated information to a second dental professional under the conditions of communication, in step d);
optionally, a third computer configured to determine the adjustment of the orthodontic appliance in step d), automatically or, preferably, with the assistance of the second dental professional;
optionally, a machine for manufacturing said orthodontic appliance;
optionally, a transport network able to transport said orthodontic appliance from said manufacturing machine to the patient.

According to a second main aspect, the invention also relates to a computer interface displaying, simultaneously on a computer screen:
an identifier of a patient; and
a timeline representing successive times that are individually selectable by an operator; and
for each selectable time selected, information relating to the dental configuration at the selected time, and preferably at least one extraoral representation of the patient's dental arches at the selected time, or “main representation”, and/or a status, at the selected time, of at least one treatment objective and/or at least one observation relating to the patient's dental configuration at the selected time; and
preferably, a treatment-objective-monitoring zone providing information elements relating to the statuses of said treatment objectives at the time when the interface is displayed;
preferably, a thread of a conversation with the patient.

The invention also relates to a computer programmed to display an interface according to the invention.

### Definitions

By “patient”, what is meant is any person, whether sick or not, undergoing an orthodontic treatment. Unless otherwise specified, the “patient” is the person for whom the conditions of communication are determined in step a). The patient is therefore to be distinguished from historical patients contributing to generation of a training dataset.

By “dental professional”, what is meant is a dentist, an orthodontist, a dental assistant, a company that manufactures orthodontic appliances, or an orthodontic laboratory.

An “orthodontic treatment” is all or part of a treatment intended to modify the dental configuration of a patient (active orthodontic treatment) or to maintain it, in particular after the end of an active orthodontic treatment (passive orthodontic treatment).

An orthodontic treatment is planned with a “treatment plan”. Thus, the “orthodontic treatment”, which designates the series of operations that in fact take place, is different from the “treatment plan”, which is the result of the design of the orthodontic treatment. The treatment plan therefore precedes the orthodontic treatment.

An orthodontic treatment may be a therapeutic or prophylactic treatment, but also an esthetic treatment.

An “orthodontic appliance” is an appliance worn or intended to be worn by a patient. An orthodontic appliance may be intended for a therapeutic or prophylactic treatment, but also an esthetic treatment. An orthodontic appliance may be in particular an archwire-and-bracket appliance, or an orthodontic aligner, or an auxiliary appliance of the Carriere Motion type. Such an aligner extends in such a way as to follow the successive teeth of the arch to which it is fastened. It defines a tray in the overall shape of a “U”. The configuration of an orthodontic appliance may in particular be determined so as to ensure it may be fastened to the teeth, but also depending on a target positioning desired for the teeth. More precisely, its shape is determined so that, in the service position, the orthodontic appliance exerts forces that tend to move the treated teeth toward their target positions (active orthodontic appliance), or to keep the teeth in this target position (passive orthodontic appliance or “retainer”).

A position or shape is said to be “abnormal” when it does not conform to a position or shape expected at the time in question, respectively.

A device such as an iPhone® is called a “mobile telephone” or “cellphone”. Such a device typically weighs less than 500 g and is equipped with a camera comprising an objective allowing it to take films or photos, or even a scanner allowing it to acquire three-dimensional digital models. A mobile telephone is further capable of exchanging data with another device more than 500 km away from the mobile telephone, and is capable of displaying said data.

By “image”, what is meant is a digital representation in two dimensions, such as a photograph or an image extracted from a film. An image is made up of pixels.

By “model”, what is meant is a three-dimensional digital model. A model consists of a set of voxels.

Generically, a set of “updated” images or an “updated” model representing, at least partially, the jaws of the patient is called an “updated representation”. A set of updated images or an updated model are preferred examples of “updated representations”.

An image or a model is “extraoral” when the acquiring device is not inserted into the patient's mouth during its acquisition.

A “representation of the arches” of the patient represents all or part of these arches, and preferably more than 2, more than 5 or more than 8 teeth, and preferably at least one portion of each arch, and preferably at least one portion of the gum of each arch.

An “angulation” is an orientation of the optical axis of an acquiring device with respect to a patient, during acquisition of an updated representation, and in particular of an image. By extension, an image is said to “have” an angulation or to be “associated with” an angulation when it was acquired with this angulation.

A “retractor”, or “dental retractor”, is a device intended to push back the lips. It comprises an upper rim and a lower rim, and/or a right-hand rim and a left-hand rim, extending around a retractor opening and intended to be introduced between the teeth and the lips. In service position, the patient's lips press against these rims, so as to make the teeth visible through the retractor opening. A retractor thus makes it possible to observe the teeth without the lips getting in the way. However, the teeth do not rest on the retractor, and hence the patient is able, by turning her or his head with respect to the retractor, to modify the teeth that are visible through the retractor opening. She or he may also modify the spacing between her or his dental arches. In particular, a retractor does not press on the teeth so as to separate the two jaws from each other. In one embodiment, a retractor is configured so as to elastically separate the upper and lower lips from each other, so as to leave the teeth visible through the retractor opening. Retractors are for example described in PCT/EP2015/074896, U.S. Pat. No. 6,923,761, or US 2004/0209225.

The “mouth closed” position is the occlusion position in which the teeth of the upper and lower arches of the patient make contact. A “mouth open” position is an open position of the mouth, in which the teeth of the upper and lower arches of the patient do not make contact.

An “updated value” is a value of an “attribute”, or “parameter”, of a shape relating to a dental configuration. It is determined from the updated representation, and preferably exclusively from the updated representation, and therefore depends on the shape of the unit formed by the patient's upper and lower jaws, in at least one relative position of the lower jaw with respect to the upper jaw, and for example in the closed-mouth position, at the update time. An updated value is, for example, a measurement on the updated representation or on the result of computer processing of the updated representation, or is a value resulting from submission of the updated representation to a neural network. It may be determined on the updated representation directly, and for example be a measurement of an overjet, or indirectly, and for example be the result of a comparison of the measurement of an overjet with a reference value, or be a status of a treatment objective, and for example be equal to a “normal overjet”. An updated value may in particular be
a dimension of the patient's dental arches,
a relative position, or a position in a frame of reference that is fixed with respect to the patient, of a point on the patient's dental arches,
a distance between two points of the patient's dental arches,
a distance between one or more points of the patient's dental arches and a frame of reference, or
a distance between a plurality of points of the same dental arch, or
a combination of one or more of said dimensions, positions and distances.

An “information element” expresses an updated value.

“Expressing” an updated value, a status for example, consists in providing a human being with information relating to this updated value, providing the status of a treatment objective for example, but also in presenting this updated value, for example in a graphical form, or in the form of a symbol or text.

An information element may be displayed on a screen, a computer or telephone screen for example. An information element therefore has a content that results from the updated value and a format that defines how this content is presented (text, font, graphic element, etc.).

The “presentation conditions” are the rules that define the format of each information element. The presentation conditions may in particular define the format of the information element depending on its recipient and/or its content and/or the communication medium used. For example, the format of an information element relating to an overbite will differ depending on whether the information element is addressed to the orthodontist or to the patient, on whether the overbite requires urgent intervention by the orthodontist or not, or on whether the information element is communicated via email or mail. The presentation conditions are preferably defined and programmed before step a). In one embodiment, they may be programmed or modified by the first dental professional in step a).

The “conditions of communication” define, for each information element, whether, and preferably to whom, said information element must be communicated in step d). They may depend on the update time and/or on the updated value associated with the information element and/or on the age of the patient and/or on the orthodontic treatment or on the type of orthodontic treatment being undergone by the patient (for example treatment with orthodontic aligners or with an archwire-and-bracket appliance or a hybrid treatment) and/or on the status of one or more other treatment objectives. They are defined in step a).

A “treatment objective” is an objective for the dental configuration of the patient, at the end of an active orthodontic treatment or at an intermediate stage (milestone) of an active orthodontic treatment. It is conventionally set by an orthodontist. For a passive orthodontic treatment, a treatment objective is an absence of modification of the dental configuration since a prior date.

The attribute of a treatment objective is called “status”. A “status” may take only a limited number of values, called “potential statuses”. At a given time, the status is equal to one of the potential statuses of the treatment objective, which is then an “updated value”. Advantageously, an information element expressing a status is easy to understand, this facilitating the decision-making after step d).

A treatment objective is “monitored” when an information element is determined in step c) for that treatment objective and may be communicated in step d), depending on the conditions of communication. The datum that sets whether the treatment objective is being monitored or not does not provide information on the patient's dental configuration at the update time and is therefore not an “updated value”.

“Updated Information” consists of a set of information elements. The information is said to be “updated” because it is determined from the updated representation acquired at the update time.

The methods according to the invention are implemented by computer, and preferably exclusively by computer, with the exception of the acquisition of the updated representation and the manufacture of the orthodontic appliances. By “computer” what is meant is any electronic device, this including a set of a plurality of machines, having information-processing capabilities. The computer may be a server remote from the patient, a cloud server for example. Preferably, the computer is a mobile telephone.

Conventionally, a computer in particular comprises: a processor; a memory; a human-machine interface, conventionally comprising a screen; and a module for communication via the Internet, via Wi-Fi, via Bluetooth® or via the telephone network. Software configured to implement a method of the invention of a step of such a method is loaded into the memory of the computer. The computer may also be connected to a printer.

“First”, “second” and “updated” are used for the sake of clarity.

“Prior” and “subsequent” refer to successive moments in time.

“Vertical”, “horizontal”, “right (-hand)”, “left (-hand)”, “front” or “from the front”, “behind”,

“above” and “below” are to be understood with reference to a patient standing vertically upright.

“Comprising” or “including” or “having” is to be interpreted non-restrictively unless indicated otherwise.

### BRIEF DESCRIPTION OF THE FIGURES

Further features and advantages of the invention will become more clearly apparent on reading the following detailed description and on studying the appended drawing, in which:

FIG. 1 schematically shows one preferred embodiment of an analyzing method according to the invention;

FIG. 2 schematically shows an interface according to the invention;

FIG. 3 shows one example of a treatment-objective-monitoring zone;

FIG. 4 shows one example of a dialog box for parameterizing a treatment objective;

FIG. 5 illustrates the monitoring of a treatment objective, in one preferred embodiment of the invention;

FIG. 6 shows one example of an updated image, taken from the front;

FIG. 7 shows one example of a system according to the invention;

FIG. 8 shows one example of an acquiring device comprising a mobile telephone and a mouthpiece.

In the various figures, identical references have been used to designate analogous or identical objects.

### DETAILED DESCRIPTION

One aim of the invention is to facilitate analysis of the dental configuration of the patient, in particular with a view to determining actions to be consequently taken, and in particular with a view to adjusting an orthodontic appliance intended for a patient.

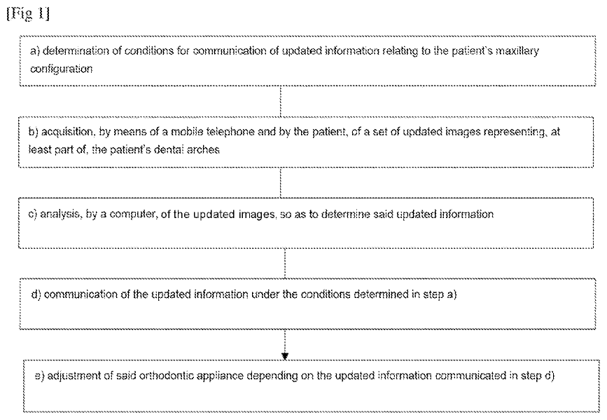

A method according to the invention is illustrated in FIG. 1. This method is described with a set of updated images by way of updated representation. However, in step b), as an alternative or in addition to the updated images, it is possible to acquire, with a portable scanner, which is preferably integrated into the patient's mobile telephone, a model of the patient's dental arches, which is referred to as the “updated model”. The analysis in step c) is adapted accordingly.

In step a), at a time referred to as the “parameterization time”, a first dental professional, preferably an orthodontist, determines, specifically for the patient, conditions for communication of updated information relating to the patient's dental configuration. These conditions of communication are then entered into a computer.

The conditions of communication determined in step a) may be chosen from a computer database containing a number of predetermined sets of conditions of communication, for example more than 2, more than 5 or more than 10, and/or less than 500 predetermined sets.

The first dental professional may optionally modify the conditions of the predetermined set that she or he has selected.

The conditions of communication determined in step a) may also be entirely defined by the first dental professional.

The parameterization time may in particular be before an orthodontic treatment or before an intermediate stage of an orthodontic treatment, and preferably less than 2 weeks, and preferably less than one week before said orthodontic treatment or said intermediate stage, respectively. The parameterization time may for example be a time scheduled for a change of orthodontic aligner or for a modification of a treatment with an archwire-and-bracket orthodontic appliance. The parameterization time may also be during a step d) of a series of steps b) to d) prior to the update time.

The first dental professional is preferably the orthodontist or dentist monitoring an orthodontic treatment that she or he has prescribed to the patient.

Preferably, at least one information element expresses a status of a treatment objective.

The conditions of communication may in particular contain one or more:
time-related conditions of communication of the information element, in particular
   one or more communication ranges, and/or
   a minimum or maximum length of time between the update time and at least one reference time, for example a time marking the end of the orthodontic treatment or of one portion of the orthodontic treatment, or the time of step d) of a prior series of steps b) to e) optionally comprising a step a), and/or
   a maximum frequency of communication of the information element, which preferably depends on the recipient of the information element; and/or
conditions regarding the recipient(s) of the information element, in particular for setting to whom the information element must be communicated.

Preferably, when a condition relates to a treatment objective, it makes reference to the status of the treatment objective at the current time, i.e. the application of the condition to the information element relating to said treatment objective leads to a different result depending on the status of the treatment objective at the update time.

In step a), the conditions for communication preferably determine, for at least one, and preferably for each information element, at least one communication range to which the update time must belong for said information element to be communicated to the second dental professional in step d). Advantageously, it is thus possible, at the parameterization time, to set the time ranges in which the information elements must be communicated.

Preferably, the communication range associated with an information element is defined depending on the recipient for whom said information element is intended.

Preferably, a communication range, and preferably any communication range, has a limited duration, preferably less than 3 months, preferably less than 2 months and/or greater than 1 week.

A communication range, and preferably any communication range, preferably starts after the parameterization time, and preferably more than one week after the parameterization time.

FIG. 5 illustrates monitoring of a treatment objective in one preferred embodiment. On an axis representing time t, a said communication range 4, a period 6 prior to the communication range 4 and a period 8 subsequent to the communication range 4 have been shown.

Monitoring of the objective starts at the parameterization time t₁. The status of the treatment objective becomes communicable to the second dental professional only from the time t₂ and only until the time t₄. In this preferred embodiment, if the status indicates, during the communication range, that the treatment objective has not been reached, it is not communicated. Preferably, if it is not reached at the time t₄, it is however communicated at the time t₄ or in the first step d) that follows the time t₄. If it is reached during the communication range 4, at the time t₃, it is communicated at this time t₃.

The interval [t₁-t₂] may be zero in the case where the second dental professional must be notified of the status of the treatment objective as soon as the parameterization has been carried out.

When the information element is a status of a treatment objective, the start of the communication range may in particular be the moment at which the first dental professional believes that the treatment objective will have been reached. The end of the communication range may in particular be the moment at which the first dental professional wishes to be notified if the treatment objective has not been reached, for example with a view to contacting the patient, for example in order to provide her or him with information and/or a recommendation and/or a new instruction in the context of the orthodontic treatment, and/or to make an appointment with her or him.

In one embodiment, in step a), the conditions for communication preferably determine, for at least one, and preferably for each information element of the updated information, a maximum frequency of communication in step d), and/or a minimum time interval since the implementation of the preceding method. Advantageously, it is thus possible to avoid communicating at a high frequency an information element that changes only slowly.

In one preferred embodiment, in step a), the conditions for communication determine, for at least one, and preferably for each information element of the updated information, the second dental professional(s) to whom the information element must be communicated. An information element may thus be transmitted, for example, exclusively to an orthodontist, or exclusively to an assistant of the orthodontist, or to both the orthodontist and the assistant. Advantageously, the orthodontist may thus decide that an information element that may be analyzed by an assistant, for example because it will not lead to a modification of the orthodontic treatment, does not need to be communicated to her or him. She or he may also decide that an information element will be communicated only depending on its value and/or on the update time. A method according to the invention thus allows the information elements to be distributed to only those persons who, at an update time, potentially have an action to perform in respect of the patient, for example modifying the orthodontic treatment, contacting the patient, preparing a new orthodontic appliance or arranging an appointment.

In particular, for a treatment objective, the choice of the second dental professional may depend on the treatment objective in question and/or the status of the treatment objective in question at the update time.

In one preferred embodiment, in step a), the conditions for communication determine, for at least one, and preferably for each information element of the updated information, whether the information element must be communicated to the patient, in particular on her or his mobile telephone, alternatively or, preferably, in addition to being communicated to the second dental professional.

In one preferred embodiment, in step a), the conditions for communication determine, for at least one, and preferably for each treatment objective, whether the treatment objective is being monitored or not.

The conditions determined in step a) allow fine filtering of the updated information, depending on its nature and/or on its value and/or on the update time. Each recipient, and in particular each second dental professional, thus receives only the information elements that concern her or him at the update time. The effectiveness of communication, and therefore the effectiveness of treatment, is thereby improved.

In particular, the information element may express a status of a treatment objective.

For example, the conditions of communication may for example make provision for:
the information element to be communicated only if the treatment objective is reached or not reached at the update time; and/or
the information element to be communicated only during a predetermined communication range; and/or
the information element to be communicated only if it has not been communicated for less than J days; and/or
the information element to be communicated to the patient and/or to a second dental professional, systematically or depending on the communication range and/or depending on its value; and/or
the information element to be systematically communicated if the update time is subsequent to a predetermined communication range or if the update time is separated from the end of a communication range by a period of a length less than a predetermined number of days.

In step b), the updated representation, preferably a set of updated images, representing the dental arches of the patient, is acquired, at the update time, by means of an acquiring device.

The update time may be
independent of any orthodontic treatment, for example so that anyone may inspect her or his dental configuration at any time, with her or his mobile telephone;
during an active orthodontic treatment;
after an active orthodontic treatment, and in particular during a passive orthodontic treatment.

The analyzing method may particularly be implemented during an active orthodontic treatment, to check its progress, the update time preferably being less than 3 months, less than 2 months, and/or more than 4 days, more than one week, and preferably more than 2weeks after an active orthodontic appliance, for example an orthodontic aligner or an orthodontic brace, intended to correct the positions of the patient's teeth has been fitted.

The analyzing method may also be implemented after an orthodontic treatment, to verify that the positions of the teeth have not changed for the worse (“relapse”). The update time is then preferably less than 6 months, less than 3 months, and/or more than 1 week, and preferably more than 2 weeks after the end of the active orthodontic treatment and fitting of a passive orthodontic appliance intended to keep the teeth in position, i.e. a so-called “retainer”.

The acquiring device is preferably selected from a mobile telephone, a so-called “connected” camera, a so-called “smart” watch, a tablet or a personal computer, optionally a laptop, comprising an image-acquiring system such as a webcam or a camera. Preferably, the acquiring device is a mobile telephone. Preferably, the acquiring device, in particular the mobile telephone, is not provided with any specific optics for acquiring updated images.

Also preferably, in order to acquire an updated image, the device for acquiring updated images is separated from the mouth of the patient by more than 5 cm, more than 8 cm, or even more than 10 cm and/or less than 50 cm. Advantageously, this distance does not need to be set precisely.

The acquiring device is used, generally remotely from any dental professional, by an operator who is the patient or a friend or relative of the patient. Preferably, the updated images are acquired by the patient.

Preferably, the updated images are acquired without the use of a holder, resting on the ground and immobilizing the acquiring device, and in particular without a tripod.

In one embodiment, however, the acquiring device is fastened to a holder that is positioned to bear against the patient's body, preferably a mouthpiece 2 partially inserted into the patient's mouth, as illustrated in FIG. 8.

Preferably, the mouthpiece 2 comprises:
a tubular retractor 2a that defines the oral opening Oo and preferably comprises a rim 3 extending radially outward, around the periphery of the oral opening Oo, and that is intended to be introduced between the lips and the teeth of the patient, and
an adapter 2b to which the mobile telephone 12 is fastened, clamped between two jaws 5₁ and 5₂ for example, as illustrated in FIG. 8, the adapter 2b being rigidly fastened to the tubular retractor 2a, preferably removably, for example by means of clips, or made of the same material as and integrally formed with the retractor, so that the objective of the mobile telephone can “see” the oral opening.

When the holder is rigid, it advantageously imposes a predetermined distance between the acquiring device and the patient's mouth. This facilitates analysis of the images updated in step c).

In one embodiment, the holder bears a conventional dental retractor.

One example of a holder is described in the European patent application filed on Oct. 10, 2017 under No. 17 306361.1.

The updated representation, in particular the updated images, are preferably extra-oral, i.e. the acquiring device is not inserted into the patient's mouth.

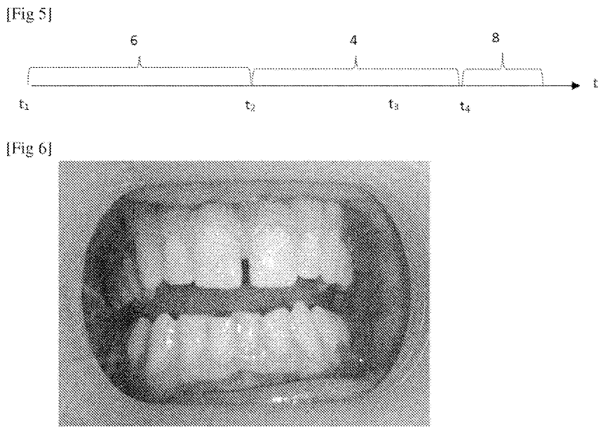

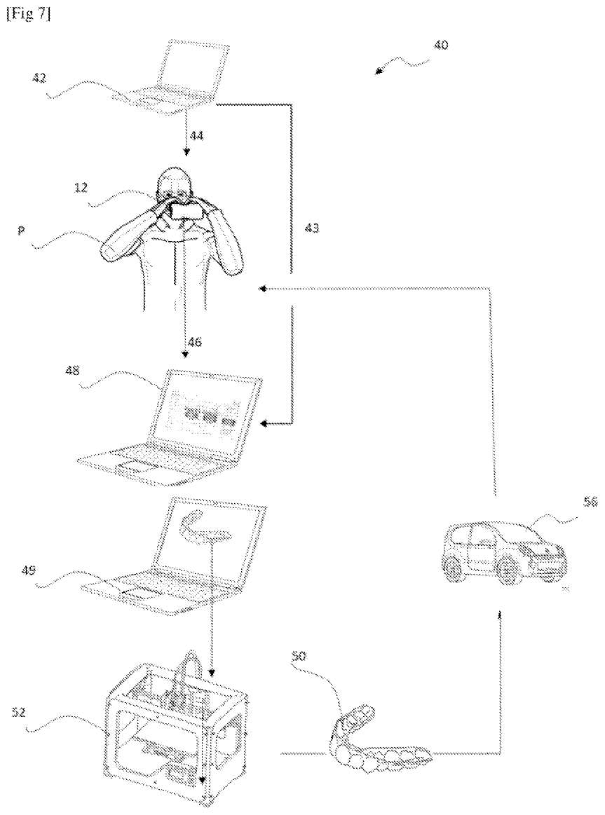

Preferably, the updated images are photographs or images extracted from a film. They are preferably in color, preferably true color. Preferably, they show the dental arches substantially as seen by the operator of the acquiring device. FIG. 6 shows one example of such an updated image, taken from the front.

In one embodiment, the patient wears a dental retractor to better expose her or his teeth. Preferably, however, no dental retractor is used. Preferably, the patient does not wear any dental retractor in step b). Of course, if necessary, the patient may have to move a cheek or a lip out of the way with a finger or with a spoon or any other utensil suitable for this purpose, for example.

The updated representation may show the arches in the “mouth open” or “mouth closed” position. Preferably, at least one updated image is acquired while the patient has her or his mouth open. Preferably, at least one updated image is acquired while the patient has her or his mouth closed.

One updated image, and preferably each updated image, shows one or more teeth and at least one portion of the patient's gum, or even lips or nose.

Also preferably, in step b), a plurality of updated images taken with different angulations, i.e. with different orientations of the acquisition device with respect to the patient's oral cavity, are acquired. For example, the set of updated images may comprise 6 images representing the dental arches “seen from the front”, “seen from the front right”, “seen from the right”, “seen from the front left”, “seen from the left” and “seen from below”, respectively.

Preferably, at least one updated image is acquired from the front of the patient (front view).

Preferably, at least one updated image is acquired from the patient's right, and at least one updated image is acquired from the patient's left.

Preferably, the operator is guided, in step b), preferably in real time, to orient the acquiring device with predetermined angulations, and/or, preferably, to take a predetermined number of images with the various angulations, and/or to orient the acquiring device with the required angulations. To this end, an application is preferably loaded into the acquiring device in order to ensure a check is embedded into step b), i.e. to check that the number and/or the angulation and/or the quality of the updated images are/is satisfactory.

The set of updated images obtained at the end of step a) preferably comprises more than two, preferably more than three, preferably more than 5, preferably more than 6 and/or less than 30, preferably less than 20, preferably less than 15, and preferably less than 10 updated images.

In step c), a computer analyzes the updated representation, preferably updated images, and determines the updated information.

Known methods for analyzing images may be used to determine an “updated” value for an attribute relating to the dental configuration of the patient at the update time, and then to generate an information element expressing this “updated” value.

Preferably, the set of updated images is submitted to a neural network trained to provide such an “updated” value.

The attribute may be quantitative, for example to define
a position of one or more points of a given dental arch or of both dental arches of the patient,
a dimension or a distance between a plurality of points of a given arch, for example an arch width, a spacing between two teeth,
a distance between a plurality of points of the two arches, for example to determine an occlusion class or the deviation of the inter-incisal midlines,
a variation over time in a said dimension, a said distance and/or a said position listed above, for example a difference between the positions of a given point of the arches at different times, or a speed of movement of said point between said times, or an acceleration of movement of said point between said times.

The attribute may be synthetic, and in particular may be a synthesis of one or more of the examples of attributes listed above. For example, it may define a level of crowding, a status of a treatment objective, an “upward”, “downward” or “stable” variation, for example in the activity of an orthodontic brace and/or in a movement of one or more teeth, or define a score resulting from a combination of a plurality of other attributes, for example with a view to assessing the complexity of the patient's dental situation.

The neural network is preferably specialized in image classification. Preferably, the neural network is a convolutional neural network (CNN), preferably one selected from the following neural networks: AlexNet (2012), ZF Net (2013), VGG Net (2014), GoogleNet (2015), Microsoft ResNet (2015), Caffe: BAIR Reference CaffeNet, BAIR AlexNet, Torch: VGG_CNN_S, VGG_CNN_M, VGG_CNN_M_2048, VGG_CNN_M_1024, VGG_CNN_M_128, VGG_CNN_F, VGG ILSVRC-2014 16-layer, VGG ILSVRC-2014 19-layer, Network-in-Network (Imagenet & CIFAR-10), Google: Inception (V3, V4).

Preferably, a squeeze-and-excitation (SE) processing block such as described by Jie Hu et al., in “Squeeze-and-Excitation Networks”, arXiv: 1709.01507v4 [cs.CV] 16 May 2019, is added to a CNN convolutional operator. Also preferably, the neural network is of the VGG type with an SE block.

To work, the neural network must conventionally be trained via a training process called deep learning, using a historical training dataset tailored to the desired function.

Conventionally, a historical training dataset is created, then the neural network is trained with this dataset.

### Historical Training Dataset

The process used to train a neural network is well known to those skilled in the art. It consists in confronting the neural network with a historical training dataset containing historical records each containing one input datum and one output datum.

The neural network thus learns to “match” the input and output data, i.e. to connect one to the other.

In order for the neural network to be able to learn to evaluate, based on a set of updated images, a value for the attribute relating to the dental configuration, the historical training dataset is preferably made up of a set of historical records each comprising:
a set of “historical” images (preferably photos) showing all the “historical” dental arches of a “historical” patient;
a historical annotation of the set of historical images, the annotation including a “historical” value of said attribute, relating to the dental configuration of the arches shown in the historical images.

Preferably, the historical training dataset contains more than 1000, more than 5 000, preferably more than 10 000, preferably more than 30 000, preferably more than 50 000, and preferably more than 100 000 historical records. The higher the number of records, the better the neural network's analytical capacity will be. The number of historical records is conventionally less than 10 000 000 or 1 000 000.

The historical records are each associated with one respective historical patient.

### Historical Images

A set of historical images is preferably similar to the set of updated images.

Preferably, the sets of historical images all contain the same number of historical images, regardless of the historical record in question. This number is preferably greater than 1, than 2, than 4, than 5 and/or less than 100, than 50, than 20, than 15 or than 10, and preferably comprised between 5 and 15.

In one embodiment, this number is 3, the 3 historical images having different angulations.

The historical images of a set of historical images are preferably acquired under conditions similar to the updated images acquired in step b). In other words, the set of updated images must be such that it could have been used as a historical record.

The number of historical images in a historical record is preferably identical to the number of updated images in the set of updated images in step a).

Preferably, the historical images are
acquired with an acquiring device of the same type as the one used for the updated images, preferably a mobile telephone if the updated images are acquired with a mobile telephone;
preferably acquired with substantially the same angulations as the updated images;
preferably color photos, preferably true-color photos if the updated images are color photos or true-color photos, respectively;
extra-oral, if the updated images are extra-oral;
preferably taken with an orientation and distance of the acquiring device with respect to the mouth substantially identical to those used when acquiring the updated images;
preferably acquired while the historical patient is wearing a dental retractor if a dental retractor is worn when acquiring the updated images;
preferably acquired while the acquiring device is fastened to a holder that is positioned to bear against the body of the historical patient, and preferably inserted into the mouth of the historical patient, if such a holder is used when the updated images are acquired.

### Historical Annotation

A historical annotation of a set of historical images includes a value for said attribute relating to the dental configuration of the historical patient.

The attribute is the same for all the historical images of the set. Preferably, the neural network is specialized in one attribute, i.e. the attribute is the same in all the historical records.

Determining the value of a historical annotation does not pose any difficulty. It may be determined by any means, for example manually or by computer, in particular by taking measurements on the historical patient or on a plaster cast of her or his teeth or on a digital three-dimensional model of the dental arch containing the tooth in question.

### Training

The neural network is then trained with the historical training dataset, by successively presenting the historical records thereto, and more precisely, for each historical record, the sets of historical images as input and the values of the attribute of the historical annotation as output.

It thus learns to deliver, as its output, when a set of images similar to a set of historical images is presented to it as input, a value for the attribute. In particular, after having been trained in this way, the neural network is able, when a set of updated images is presented to it as input, deliver as output an “updated” value for the attribute, and thus provide information on the dental configuration of the arches shown in the updated images.

After training, the set of updated images generated in step b) is input into the neural network. In response, the neural network delivers a value for the attribute, which is referred to as the “updated value”.

In one preferred embodiment, a plurality of neural networks is trained, each specialized in one attribute. Preferably, a sufficient number of updated images is then acquired in step b) to form sets of “specialized” updated images appropriate for each of the specialized neural networks.

The neural network may use a dental-arch model instead of a set of images. The neural network is trained in a similar way to that described above, the sets of historical and updated images being replaced by historical and updated models, respectively.

Before an information element associated with an updated value is prepared, the updated value may be processed, and for example replaced by the result of the difference between the updated value and a reference value, or be combined with other updated values. For the sake of clarity, the result of such processing will be called the “derived” updated value.

In the context of monitoring an orthodontic treatment, the reference value may be the value expected for the attribute at the update time or at the end of the orthodontic treatment, optionally with a margin of tolerance. Irrespective of any processing, a reference value may for example be a value considered to be normal for the attribute, for example at the update time.

In one embodiment, the reference value is patient-independent, i.e. applicable to any patient of a group of patients. It thus constitutes a standard. Preferably, the standard is specific to a pathology and/or a type of orthodontic treatment, and/or to a group of patients sharing a common characteristic, for example the same age class and/or the same sex. In particular, the standard may define a dental configuration at the end of a treatment or at the update time.

An, optionally derived, updated value is presented, in step d), under the conditions of communication, in the form of a corresponding information element. In step c), the information element is determined, by computer and under representation conditions, from the, optionally derived, updated value.

In one embodiment, in step c), the updated information results from analysis of the updated images alone, optionally using one or more reference values.

In one embodiment, an information element is dependent on the intended recipient for whom it is intended and/or on the updated value.

For example, if the updated value indicates that the orthodontic treatment is not proceeding as it should, the corresponding information element intended for the patient may be text providing a reminder of the need to follow the orthodontist's instructions or asking her or him to make an appointment. The corresponding information element intended for an assistant of the orthodontist may be a purchase order for a new orthodontic aligner or for a new dental brace. The corresponding information element intended for the orthodontist may be a medical datum. If the updated value indicates that the orthodontic treatment is not proceeding as it should, the information element may differ depending on the severity of the situation. For example, if the situation is serious, the information element intended for the patient may be text in red indicating with insistency the need to make an appointment very quickly. If the situation is not serious, the information element intended for the patient may be text in black indicating, for information purposes, that the treatment has deviated slightly from the treatment plan.

Of course, if knowledge of the conditions of communication is required to prepare an information element, the computer that prepares the information elements is informed of said conditions of communication. For example, if the content and/or the format of an information element depend/depends on its recipient, these conditions make it possible to know who the recipients of the communication in step d) are to be, for example depending on the updated value and/or the update time, and therefore to prepare the information element accordingly.

In one embodiment, the updated information takes the form of a message configured for its recipient. Preferably, the message is generated by computer, preferably selected from a database of standard messages, and preferably configured for the recipient. In one embodiment, it is checked, and optionally modified by a dental professional before being presented in step d).

In particular, if an information element must be communicated to the patient, preferably on her or his mobile telephone, it preferably takes the form of a message specifically configured for the patient. The communication of the information element thus differs depending on whether the information element is addressed to a second dental professional or to the patient.

Preferably, a message addressed to the patient includes
a recommendation, for example to make an appointment with an orthodontist or to modify an orthodontic treatment being undergone; and/or
an instruction, for example regarding the date of the next step b) to be carried out; and/or
a medical opinion on her or his dental configuration; and/or
information on the orthodontic treatment being undergone by the patient, for example photos, preferably annotated photos, and/or statistics and/or information allowing comparison with a prior dental configuration; and/or
encouragement, for example taking the form of “credit marks” awarded depending on compliance with the orthodontic treatment and giving rise to a reward; and/or
statistics showing the progress due to compliance with the orthodontic treatment and/or the advance of the orthodontic treatment.

Preferably, a message addressed to a second dental professional includes an instruction, for example, to make an appointment with the patient, to contact another dental professional, or to modify an orthodontic appliance intended for the patient.

In one embodiment, the, optionally derived, updated value defines a status of a treatment objective. A method according to the invention is in particular well suited to concise communication in respect of treatment objectives.

Preferably, the status of a treatment objective is selected from a group consisting of less than 10, preferably less than 5, and preferably less than 4 potential statuses.

In one preferred embodiment, the status of a treatment objective, and preferably of any treatment objective, is selected from a group consisting of 3 potential statuses. It preferably specifies whether the treatment objective has been met, has not been met but is in compliance with the treatment plan, or has not been met and is not in compliance with the treatment plan. In one embodiment, the status of a treatment objective, and preferably of any treatment objective, is selected from a group consisting of 2 potential statuses. Preferably, it specifies whether or not the treatment objective has been reached at the update time.

Advantageously, a limited number of potential statuses allows the status to be very concise.

Preferably, the information element relating to the status of a treatment objective has a different format (color, character size, addition of a symbol, etc.) depending on the value of the status, and for example depending on whether the treatment objective has been reached, has not been reached but is in compliance with the treatment plan, or has not been reached and is not in compliance with a treatment plan. This conditional formatting advantageously allows the dental professional, in step d), to immediately identify the treatment objectives on which she or he must focus her or his attention, and thus, indirectly, accelerates the process of possible adjustment of the orthodontic treatment, and in particular the production of a new orthodontic appliance.

Preferably, the updated information determined in step c) specifies a status, at the update time, of more than 1, more than 2, more than 5, and/or less than 20 or less than 15 treatment objectives.

Preferably, for at least one, and preferably for each treatment objective, the updated information contains an evaluation of the time remaining, from the update time, to reach the treatment objective, preferably in compliance with a treatment plan established before the update time, preferably at the parameterization time, and/or contains an indication of the date since which the treatment objective has been monitored. The second dental professional may thus prioritize the examination of the treatment objectives that must be reached quickly. If time is short, she or he may ignore the others and postpone their analysis to an update time in a subsequent series of steps b) to e).

Preferably, the one or more treatment objectives are selected from the following group:
the occlusion of the canines of the patient has reached Class I,
the occlusion of the molars of the patient has reached Class I,
the gaps in the anterior sector of the patient have closed,
the gap resulting from extraction of a tooth has closed,
the overjet of the patient has reached a normal level, and is preferably comprised between 1 and 3 mm,
the overbite of the patient has reached a normal level, and is preferably comprised between 1 and 3 mm,
the inter-incisal midlines of the maxillary and mandibular arches are not offset,
the median lines of the lower and upper arches are not offset,
the patient does not have a lateral offset of the upper arch with respect to the lower arch,
the orthodontic brace and/or the auxiliary appliances are passive;
no or few teeth are detected to have moved between the last two inspections of the maxillary and/or mandibular arch, preferably no teeth being detected to have moved,
all the deciduous teeth have fallen out,
absence of lateral open bite (lateral open bite closed),
absence of posterior open bite,
absence of anterior open bite,
absence of anterior crossbite,
absence of posterior crossbite,
improvement in crowding,
absence of gingivitis,
absence of tartar,
improvement in hygiene,
stabilization of recession,
sufficient cleaning of the orthodontic appliance,
diastemata closed,
absence of black triangles,
absence of infection (aphthae for example) or irregularities in the mucosa,
improvement of the shade/whiteness of the teeth.

The following table provides examples in respect of monitoring treatment objectives, the information elements taking the form of traffic lights:

TABLE 1

| | | Potential | Information |
|---|---|---|---|
| Treatment objective | Attribute | statuses | elements |
| Canine occlusion in Class I | Occurrence of canine occlusion in Class I | True/False | Green/amber/red |
| | | | light⁽¹⁾ |
| Molar occlusion in Class I | Occurrence of molar occlusion in Class I | True/False | Green/amber/red |
| | | | light⁽¹⁾ |
| Anterior gaps closed | Occurrence of closure of anterior gaps | True/False | Green/amber/red |
| | | | light⁽¹⁾ |
| Any gap resulting from extraction | Occurrence of closure of all gaps resulting | True/False | Green/amber/red |
| of a tooth closed | from tooth extraction | | light⁽¹⁾ |
| Normal overjet | Occurrence of normal overjet | True/False | Green/amber/red |
| | | | light⁽¹⁾ |
| Normal overbite | Occurrence of normal overbite | True/False | Green/amber/red |
| | | | light⁽¹⁾ |
| Median lines of the upper and lower | Alignment of the median lines of the lower | True/False | Green/amber/red |
| arches aligned | and upper arches | | light⁽¹⁾ |
| Absence of lateral offset of the upper | Occurrence of a lateral offset of the upper | True/False | Green/amber/red |
| arch with respect to the lower arch | arch with respect to the lower arch | | light⁽¹⁾ |
| No movement of a tooth between the last | No occurrence of movement of a tooth between | True/False | Green/amber/red |
| two inspections | the last two inspections | | light⁽¹⁾ |
| Absence of deciduous teeth | Presence of a deciduous tooth | True/False | Green/amber/red |
| | | | light⁽¹⁾ |

⁽¹⁾green: objective met; orange: objective not met but allowed time not exceeded; red: objective not met and allowed time exceeded

The granularity of these treatment objectives is well suited to a rapid analysis of the patient's dental configuration.

Preferably, the light has a different color, for example gray, if the treatment objective is not being monitored. In this case, it does not provide any information on the patient's dental configuration at the update time and is therefore not an information element expressing a status of the treatment objective.

Step c) therefore leads to a set of information elements to be displayed, which information elements are determined depending on the analysis of the updated representation and on the conditions determined in step a).

In step d), the updated information determined in step c) is communicated to at least one second dental professional, under the conditions determined in step a).

The second dental professional may be identical to or different from the first dental professional. In particular, she or he may be an assistant of the first dental professional.

Each treatment objective preferably belongs to a sub-group defined depending on a type of movement targeted by the treatment objective. The sub-group is preferably chosen from the following sub-groups: “antero-posterior” sub-group, “transverse” sub-group, “vertical” sub-group and “other” sub-group. Preferably, in step d), the updated information presented on the screen groups the information elements relating to the treatment objectives in zones of the screen that are specific to the sub-groups to which they belong. Advantageously, this grouping facilitates communication of the updated information to the second dental professional in step d).

The interface for presenting the updated information is preferably presented on a screen of a PC-type computer or on a portable screen, for example on a screen of a mobile telephone.

Preferably, this interface displays, for at least one treatment objective, and preferably for each treatment objective, a virtual button the activation of which generates a dialog allowing modification of the conditions of communication, and in particular of a communication range and/or of a list of recipients for this treatment objective. Preferably, the interface is also configured to allow modification of a criterion to be used in the analysis in step c), of a reference value for example.

Preferably, the interface also displays a thread of a conversation with the patient, and in particular between the patient on the one hand and the first dental professional and/or the second dental professional on the other hand. Advantageously, the conversation thread allows the second dental professional to better understand the choices made before the update time in respect of the orthodontic treatment of the patient, and therefore to tailor her or his decision accordingly.

Preferably, the conversation thread is used to display the information elements intended for the patient.

Preferably, the user of the interface may participate in the conversation thread, directly from the interface. Advantageously, she or he may thus interrogate the patient very effectively.

Preferably, at least some of the communication in step d) is carried out by means of an interface configured to implement step a).

Preferably, in step d), the first and/or second dental professional(s) redefine/redefines at least some of the conditions of communication, with a view to subsequent execution of other series of steps b) to e).

In one embodiment, the orthodontic treatment and/or one or more treatment objectives are modified based on the updated information communicated in step d).

In step e), subsequent to step d), depending on the updated information communicated, an orthodontic appliance intended for the patient is either adjusted or not.

The second dental professional may, based on the updated information, consider that no adjustment is necessary or that an adjustment is necessary. In case of adjustment, she or he may use the updated information and/or another source of information. In particular, she or he may carry out additional measurements on the patient's teeth.

The adjustment may consist in manufacturing all or some of a new orthodontic appliance, for example a new orthodontic aligner, or in changing to a new dental brace, or in adjusting an existing orthodontic appliance, for example an archwire-and-bracket orthodontic appliance.

In step e), an appointment may be made between the patient and the first and/or the second dental professional, and/or the modified orthodontic appliance may be sent to the patient.

Preferably, steps b) to e) are implemented cyclically, after a step a) preferably executed before the start of an orthodontic treatment.

Preferably, the series of steps b) to e) is carried out a number of times, preferably at regular intervals. The time interval between two series is preferably greater than 1 week, preferably greater than 2 weeks, and/or less than 2 months, and preferably less than 1 month.

Preferably, at least some of the update times of the various series are determined before the start of the orthodontic treatment. Preferably, they correspond to times at which a modification or change of an orthodontic appliance intended for the patient is planned.

Preferably, before each update time, a notification is sent to the patient to remind her or him of the need to acquire an updated representation. Preferably, the notification indicates a date before which the patient must perform this acquisition, this date preferably being less than 2 days from the date when the notification was sent.

The notification may be delivered in paper form or preferably in electronic form, preferably to the mobile telephone, for example in the form of an email, an automatic alert from a specialized mobile application or an SMS. Such a reminder may be sent by the orthodontic practice or laboratory or by the first and/or second dental professional(s) or by a specialized application loaded onto the mobile telephone. The notification may be sent systematically. It is preferably only sent if the patient has not already acquired said updated representation.

Preferably, a number of said notifications are sent successively, until the user acknowledges receipt of the notification or has acquired the requested updated representation.

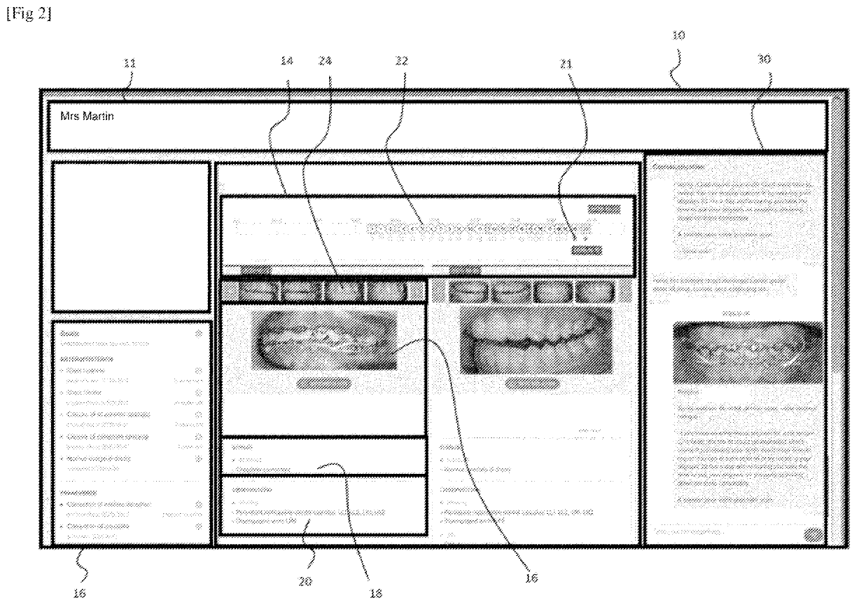

FIG. 2 shows an interface 10, particularly intended for steps a) and d).

The interface simultaneously presents on a computer screen:
an identifier of a patient, in the identification zone 11; and
a timeline 14 representing successive times that are individually selectable by an operator;
for each selectable time selected,
   at least one, preferably extraoral, representation of the patient's dental arches at the selected time, or “main representation” 16 and/or
   a status 18, at the selected time, of at least one treatment objective, and preferably only the one or more statuses of the treatment objectives that, under the conditions of communication valid at the selected time, had to be communicated at this selected time and/or
   at least one observation 20 relating to the patient's dental configuration at the selected time; and
preferably a treatment-objective-monitoring zone 32 containing the information elements relating to the statuses of the treatment objectives of the last step d) carried out.

In particular, the identification zone 11 may give the name and/or show a photo of the patient.

The timeline 14 may start before the initial time marking the start of an orthodontic treatment and extend to after the final time marking the end of an orthodontic treatment. Preferably, it extends from the initial time marking the start of an orthodontic treatment and at least up to an update time of a step a) of a method according to the invention, it preferably not extending beyond said update time.

It shows a succession of times that may each be selected. The selection of a time, achieved by clicking on the circle 21 symbolizing this time, makes it possible to view a representation of the patient's dental arches and/or an information element relating to a treatment objective and/or an observation relating to the patient's dental configuration at that time. By selecting a time on the timeline, the operator may thus advantageously access relevant information to assess the dental situation at that time.

Preferably, a number of times may be selected simultaneously. Thus, the operator may advantageously compare relevant information to assess the dental situation at the various selected times. A selected time may in particular be an update time of a step b). Preferably, the update time of the last step b) performed is selected by default.

The timeline is preferably linear, i.e. the distance between the representations of two times on the timeline is proportional to the time interval between these two times.

The timeline preferably shows, for each selectable time, an associated symbol 22 relating to the patient's dental configuration at said selectable time, and preferably relating to the advancement of the orthodontic treatment with respect to the treatment plan. Advantageously, the operator may thus immediately see periods during which the dental situation had a particular property, and for example see periods during which the orthodontic treatment progressed in compliance with the treatment plan and those when this was not the case. The operator may thus very quickly locate periods that she or he thinks useful to her or his analysis.

In one embodiment, the nature of the information underlying the symbols 22 of the timeline may be modified by the operator. For example, the operator may select one treatment objective so that the symbols illustrate compliance or non-compliance of the orthodontic treatment with regards to the treatment plan considering only this treatment objective.

Preferably, any symbol on the timeline is selected from a group containing less than 10 and preferably less than 5 values, and preferably less than 4 or less than 3 potential (i.e., selectable) symbols. In one preferred embodiment, the symbol is selected from a group containing 2 potential symbols indicating whether the orthodontic treatment is progressing in compliance with the treatment plan or not, generally or for a selected treatment objective, respectively.

For at least one, and preferably for each selected time on the timeline, the interface preferably displays at least one observation on the dental situation of the patient, the way in which said observation is displayed preferably depending on the importance of said observation to the orthodontic treatment being undergone by the patient.

The main representation 16 is preferably an image, preferably a photo, preferably a true-color photo, taken, at the selected time, by the patient, preferably with her or his mobile telephone, preferably extra-orally.

Preferably, for at least one, and preferably for each selected time on the timeline, the interface 10 displays a plurality of thumbnails 24 each showing extraoral representations of the patient's dental arch at the selected time, preferably with different angulations, each thumbnail being individually selectable by the operator, the main representation 16 being an enlarged view of the selected thumbnail.

Preferably, each thumbnail 24 has dimensions (length and width) at least two times, and preferably at least three times, smaller than those of the main representation 16. Each thumbnail is preferably an image, preferably a photo, preferably a true-color photo, taken, at the selected time, by the patient, preferably with her or his mobile telephone, preferably extra-orally, preferably in the same step b).

Preferably, all the times simultaneously selected on the timeline are processed identically, in particular in order to display, each time, for the time in question, a main representation 16 and preferably thumbnails 24, and/or an information element for at least one treatment objective, and/or at least one observation on the patient's dental configuration.

More preferably, the interface 10 displays a thread 30 of a conversation with the patient, and in particular between the patient and the operator.

Also preferably, the interface 10 displays general information about the treatment, in particular a start date of the treatment and/or the date of the next acquisition of updated images by the patient.

Preferably, any object shown on the interface (symbols and/or thumbnails in particular) may be selected by clicking on the object.

An interface 10 according to the invention may advantageously be implemented in step d) of communication of the updated information, the operator being the second dental care professional.

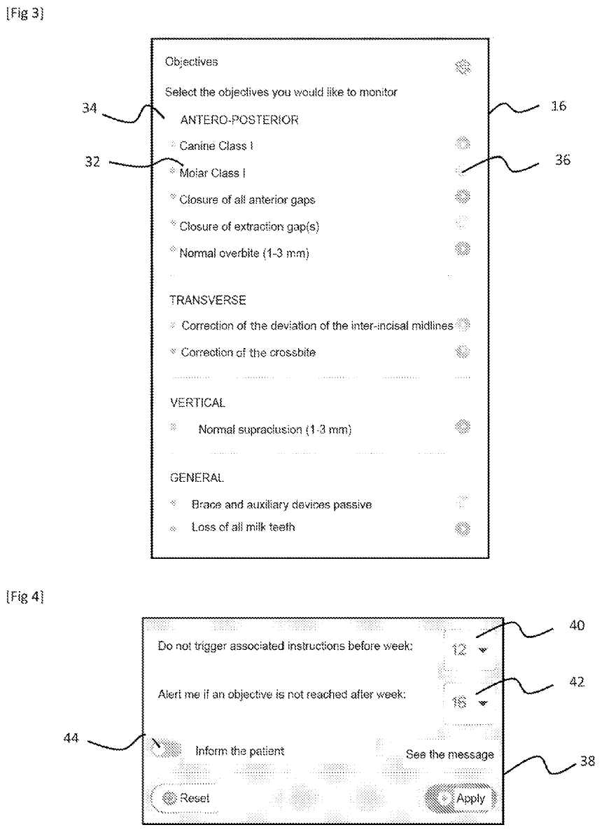

It preferably comprises an objective-monitoring zone 32, shown in greater detail in FIG. 3, which displays the information elements for the various treatment objectives determined in the last execution of a step c).

The objective-monitoring zone 32 lists all the treatment objectives 34, grouped into said sub-groups 34.

Each treatment objective is associated with a tag taking the form of a dot, the color of which indicates the status of the treatment objective, preferably as detailed in the following list:
gray: this treatment objective is not being monitored;
blue: the treatment objective is being monitored, has not yet been reached, and the deadline for reaching this treatment objective has not been reached;
green: the treatment objective has been reached;
red: the treatment objective is being monitored, has not yet been reached, and the deadline for reaching this treatment objective has been exceeded.

A virtual button 36 is associated with each treatment objective. Pressing this button causes the dialog box shown 38 in FIG. 4 to open. The operator may then specify a start date 40 and an end date 42 for a communication range and whether the patient must be informed of the status of the objective, by moving the cursor 44.

### System

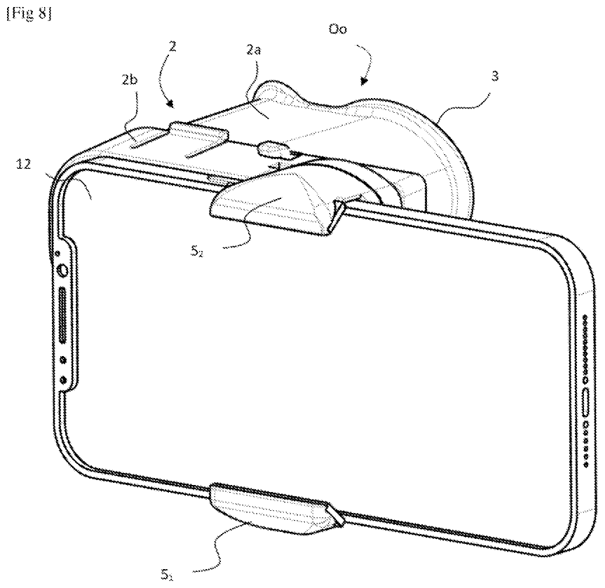

FIG. 7 illustrates one example of a system according to the invention and illustrates the implementation of a method according to the invention:

This system 40 comprises:
preferably a first computer 42, parameterized by the first dental professional to define the conditions of communication 43, and preferably configured to send the patient P a reminder 44 so that she or he acquires an updated representation 46 with a mobile telephone 45, in the present case one fastened to a mouthpiece of the type shown in FIG. 8;
the mobile telephone 45, configured to acquire said updated representation 46;
a second computer 48, identical to or different from the first computer 42, preferably remote from and in communication with the mobile telephone 45,
   able to receive and analyze said updated representation 46 from the mobile telephone to determine updated information,
   able to receive the conditions of communication 43 of the first computer 42, and
   able to present the updated information to a second dental professional under the conditions of communication 43;
a third computer 49 able to design or modify, preferably with the assistance of the second dental professional, an orthodontic appliance, for example an orthodontic aligner 50;
optionally, a machine 52 for manufacturing the orthodontic appliance, for example able to manufacture the orthodontic aligner 50 designed with the third computer, preferably from a model generated by said third computer;
preferably a transport network 56 able to transport the orthodontic appliance, for example the orthodontic aligner 50, from said manufacturing machine to the patient P.

As should now be clear, the invention provides a solution that improves the effectiveness of communication with the one or more second dental professionals. The analysis of the dental situation is improved, and therefore the adjustment of the orthodontic appliance is more exact.

In particular, the invention may be used at least to:
monitor a movement of the lower jaw with respect to the upper jaw, and/or
detect an abnormal position of the lower jaw with respect to the upper jaw, and/or
monitor a change in the shape of the lower jaw and/or the upper jaw, and/or
detect an abnormal shape of the shape of the lower jaw and/or the upper jaw.

Of course, the invention is not limited to the embodiments described in detail above.

1. A method for adjusting an orthodontic appliance intended for a patient, said method comprising the following steps:
a) at a parameterization time, determination, by a first dental professional and specifically for said patient, of conditions for communication of updated information relating to the patient's dental configuration;
b) at an update time subsequent to the parameterization time, acquisition, by means of an acquiring device and by the patient or a friend or relative of the patient, of an extraoral representation of at least one portion of the patient's dental arches, which is referred to as the “updated representation”, said updated representation including a set of images and/or of a three-dimensional digital model;
c) analysis of said updated representation, so as to determine information on said dental configuration at the update time, or “updated information”, the updated information including a set of information elements each expressing a status of a respective treatment objective, with regard to the updated representation; and
d) communication of the updated information to at least one second dental professional, under the conditions determined in step a).

2. The method as claimed in claim 1, wherein the conditions determined in step a) set for at least one said treatment objective,
at least one communication range, exclusively during which the corresponding information element, determined in step c), is communicable in step d); and/or
a maximum frequency of communication of the information element.

3. The method as claimed in claim 2, wherein the conditions determined in step a) set, for said treatment objective, different said communication ranges depending on the status of said treatment objective.

4. The method as claimed in claim 3, wherein the conditions determined in step a) set, for said treatment objective, a first said communication range for the information element expressing that the treatment objective has been reached, and, immediately after said first communication range, a second said communication range for the information element expressing that the treatment objective has not been reached.

5. The method as claimed in claim 1, wherein the conditions of communication of at least one information element determined in step a) are dependent on the treatment objective in question and/or on the status of the treatment objective expressed by said information element and/or on the recipient of the information element.

6. The method as claimed in claim 1, wherein the status of a treatment objective, is selected from a group of potential statuses containing less than 10 potential statuses.

7. The method as claimed in claim 1, wherein the number of treatment objectives is less than 30.

8. The method as claimed in claim 1, wherein, for at least one treatment objective, the updated information contains an evaluation of the time remaining, from the update time, to reach the treatment objective in compliance with a treatment plan established before the update time, and/or contains an indication of the date since which the treatment objective has been monitored.

9. The method as claimed in claim 1, wherein, in step c), the updated representation is submitted to at least one convolutional neural network.

10. The method as claimed in claim 1, wherein the acquiring device is a mobile telephone and the updated representation including a set of images, referred to as “updated images”.

11. The method as claimed in claim 1, wherein at least one second dental professional is different from the first dental professional.

12. The method as claimed in claim 1, wherein the parameterization in step a) and the communication in step d) are carried out by means of the same computer interface.

13. The method as claimed in claim 12, wherein the interface simultaneously presents:
an identifier of the patient; and
a timeline representing successive times that are individually selectable by an operator; and
for each selectable time selected, at least one extraoral representation of the patient's dental arches at the selected time, or “main representation”, and/or an information element expressing a status, at the selected time, of at least one treatment objective and/or at least one observation relating to the patient's dental configuration at the selected time; and
a treatment-objective-monitoring zone providing information elements expressing statuses of said treatment objectives at the time when the interface is displayed.

14. The method as claimed in claim 1, comprising, after step d), the following step:
e) depending on the updated information communicated in step d), either adjusting said orthodontic appliance or not.

15. The method as claimed in claim 1, wherein series of steps b) to d), or, when the preceding claim applies, series of steps b) to e), are repeated at various update times during orthodontic treatment of a patient.

16. The method as claimed in claim 15, wherein the conditions determined in step a) set for at least one said treatment objective,
for each step c) of a series of steps b) to d), or, when the preceding claim applies, of a series of steps b) to e), at least one communication range, exclusively during which the corresponding information element, determined in said step c), is communicable in step d) of said series; and/or
a maximum communication frequency of the information element, said frequency being independent of the times at which the acquisition steps b) are carried out.

17. The method as claimed in claim 1, wherein said treatment objective is selected from the following group:
membership of the occlusion to a predefined occlusion class, each occlusion class defining an inter-arch relationship in the sagittal plane,
gaps between the teeth, of the patient, i.e. diastemata, less than predefined gaps,
gap resulting from the extraction of a tooth of the patient less than a predefined gap,
overjet less than a predefined overjet,
overbite less than a predefined overbite,
offset of the inter-incisal midline of the maxillary and mandibular arches less than a predefined offset,
offset of the median lines of the lower and upper arches of the patient less than a predefined offset,
lateral offset of the upper arch with respect to the lower arch less than a predefined offset,
level of activity of the orthodontic appliance less than a predefined level,
rate of change of the movements of the teeth less than a predefined level,
amplitude of a movement of one or more teeth of the maxillary and/or mandibular arch of the patient, from where they were on prior inspection, less than a predefined amplitude,
correction of the rotation of one or more teeth achieved,
alignment of the teeth of an arch in the occlusal plane achieved,
levelling of the teeth of an arch achieved,
level of abrasion of one or more teeth less than a predefined level,
the number of deciduous teeth of the patient that have fallen out less than a predefined number,
level of lateral open bite less than a predefined level,
level of posterior open bite less than a predefined level,
level of anterior open bite less than a predefined level,
absence of anterior crossbite,
absence of posterior crossbite,
crowding less than predefined crowding,
absence of gingivitis,
absence of peri-implantitis,
amount of tartar less than a predefined amount,
a measure of hygiene level greater than a predefined measure of hygiene level,
recession value within a predefined range,
level of cleanliness of the orthodontic appliance greater than a predefined level,
gap closure or diastema correction less than a predefined level,
absence of black triangles,
absence of infection, in particular absence of aphthae, or irregularities in the mucosa,
shade/whiteness value of the teeth within a predefined range.

18. A system for implementing a method for adjusting an orthodontic appliance intended for a patient, said method including the following steps:
a) at a parameterization time, determination, by a first dental professional and specifically for said patient, of conditions for communication of updated information relating to the patient's dental configuration;
b) at an update time subsequent to the parameterization time, acquisition, by means of an acquiring device and by the patient or a friend or relative of the patient, of an extraoral representation of at least one portion of the patient's dental arches, which is referred to as the “updated representation”, said updated representation including a set of images and/or of a three-dimensional digital model;
c) analysis of said updated representation, so as to determine information on said dental configuration at the update time, or “updated information”, the updated information including a set of information elements each expressing a status of a respective treatment objective, with regard to the updated representation;
d) communication of the updated information to at least one second dental professional, under the conditions determined in step a),
e) optionally, depending on the updated information communicated in step d), either adjusting the orthodontic appliance or not,
said system comprising:
a first computer, parameterized, in step a), by the first dental professional to define the conditions of communication of the updated information;
an acquiring device, configured to acquire said updated representation in step b);
a second computer, identical to or different from the first computer
able to receive and analyze said updated representation from the acquiring device so as to determine updated information in step c),
able to receive the conditions of communication from the first computer, and
able to present the updated information to a second dental professional under the conditions of communication, in step d).

19. The system as claimed in claim 18, wherein the conditions determined in step a) set for at least one said treatment objective,
at least one communication range, exclusively during which the corresponding information element, determined in step c), is communicable in step d); and/or
a maximum frequency of communication of the information element.

20. The system as claimed in claim 19, wherein the conditions determined in step a) set, for said treatment objective, different said communication ranges depending on the status of said treatment objective.

21. The system as claimed in claim 20, wherein the conditions determined in step a) set, for said treatment objective, a first said communication range for the information element expressing that the treatment objective has been reached, and, immediately after said first communication range, a second said communication range for the information element expressing that the treatment objective has not been reached.

22. The system as claimed in claim 18, wherein the conditions of communication of at least one information element determined in step a) are dependent on the treatment objective in question and/or on the status of the treatment objective expressed by said information element and/or on the recipient of the information element.

23. The system as claimed in claim 18, wherein the status of a treatment objective, is selected from a group of potential statuses containing less than 10 potential statuses.

24. The system as claimed in claim 18, wherein the number of treatment objectives is less than 30.

25. The system as claimed in claim 18, wherein, for at least one treatment objective, the updated information contains an evaluation of the time remaining, from the update time, to reach the treatment objective in compliance with a treatment plan established before the update time, and/or contains an indication of the date since which the treatment objective has been monitored.

26. The system as claimed in claim 18, wherein the second computer includes a convolutional neural network configured to analyze said updated representation (46) so as to determine updated information in step c).

27. The system as claimed in claim 18, wherein the acquiring device is a mobile telephone and the updated representation including a set of images, referred to as “updated images”.

28. The system as claimed in claim 18, wherein the first and second computers have the same computer interface for the parameterization in step a) and the communication in step d).

29. The system as claimed in claim 29, wherein the interface simultaneously presents:
an identifier of the patient; and
a timeline representing successive times that are individually selectable by an operator; and
for each selectable time selected, at least one extraoral representation of the patient's dental arches at the selected time, or “main representation”, and/or an information element expressing a status, at the selected time, of at least one treatment objective and/or at least one observation relating to the patient's dental configuration at the selected time; and
a treatment-objective-monitoring zone providing information elements expressing statuses of said treatment objectives at the time when the interface is displayed.

30. The system as claimed in claim 18, comprising,
a third computer (49), identical to or different from the first and second computers, configured to, depending on the updated information communicated in step d), determine the suitability of the orthodontic appliance in step d), automatically or with the assistance of the second dental professional; and/or
a machine (52) for manufacturing said orthodontic appliance;
optionally, a transport network (56) able to transport said orthodontic appliance (50) from said manufacturing machine to the patient (P).

31. The system as claimed in claim 18, wherein said treatment objective is selected from the following group:
membership of the occlusion to a predefined occlusion class, each occlusion class defining an inter-arch relationship in the sagittal plane,
gaps between the teeth of the anterior sector of the patient less than predefined gaps,
gap resulting from the extraction of a tooth of the patient less than a predefined gap,
overjet less than a predefined overjet,
overbite less than a predefined overbite,
offset of the inter-incisal midline of the maxillary and mandibular arches less than a predefined offset,
offset of the median lines of the lower and upper arches of the patient less than a predefined offset,
lateral offset of the upper arch with respect to the lower arch less than a predefined offset,
level of activity of the orthodontic appliance less than a predefined level,
rate of change of the movements of the teeth less than a predefined level,
amplitude of a movement of one or more teeth of the maxillary and/or mandibular arch of the patient, from where they were on prior inspection, less than a predefined amplitude,
correction of the rotation of one or more teeth achieved,
alignment of the teeth of an arch in the occlusal plane achieved,
levelling of the teeth of an arch achieved,
level of abrasion of one or more teeth less than a predefined level,
the number of deciduous teeth of the patient that have fallen out less than a predefined number,
level of lateral open bite less than a predefined level,
level of posterior open bite less than a predefined level,
level of anterior open bite less than a predefined level,
absence of anterior crossbite,
absence of posterior crossbite,
crowding less than predefined crowding,
absence of gingivitis,
absence of peri-implantitis,
amount of tartar less than a predefined amount,
a measure of hygiene level greater than a predefined measure of hygiene level,
recession value within a predefined range,
gap closure or diastema correction less than a predefined level,
diastema level less than a predefined level,
absence of black triangles,
absence of infection, in particular absence of aphthae, or irregularities in the mucosa,
shade/whiteness value of the teeth within a predefined range.
